(19) United States
(12) Patent Application Publication
(10) Pub. No.: US 2017/0239285 A1
(43) Pub. Date: Aug. 24, 2017

(54) DEVELOPMENT OF MAGNETIC-MICRO PARTICLES THAT PROVIDE CONTROLLED GLYCOSAMINOGLYCAN (GAG) RELEASE AND THE INTRAVESICAL USAGE OF IT IN INTERSTITIAL CYSTITIS

(71) Applicant: Bülent EROL, Istanbul (TR)

(72) Inventor: Bülent EROL, Istanbul (TR)

(21) Appl. No.: 15/511,166

(22) PCT Filed: Aug. 14, 2015
(86) PCT No.: PCT/TR2015/050070

§ 371 Date: Mar. 14, 2017

(30) Foreign Application Priority Data
Sep. 17, 2014 (TR) 2014/10933

(51) Int. Cl. A61K 31/726; A61K 9/48; A61K 9/00; A61K 9/16; A61K 9/50; A61K 41/00

(52) U.S. Cl. CPC A61K 31/726; A61K 9/5026; A61K 41/0028; A61K 9/0034; A61K 9/167; A61K 9/5089; A61K 9/485

(57) ABSTRACT

Disclosed is the production of Glycosaminoglycan covered with magnetic nanoparticles, with nanotechnology to be used in the repair of Glycosaminoglycan layer that is damaged in the bladder due to interstitial cystitis.

### TECHNICAL FIELD

The application field of the invention is Urology-Neurourology under Surgical Medical Sciences title.

The invention especially relates to interstitial cystitis minimal invasive treatment field. With the present invention, the damaged glycosaminoglycan layer in bladder due to cystitis is constantly repaired.

### STATE OF THE ART

Interstitial Cystitis is a disease which does not have a certain treatment yet. The most important reason for this is the defect of the Glycosaminoglycan layer that covers the inner surface of the bladder and the degeneration created on the inner surface of the bladder by the urine with toxic effect. At the present time although the most effective treatment seems to be intravesical drug use alongside oral treatment, the benefit of this treatment is limited and it has two disadvantages. The first one is the necessity of application in the bladder via probe or urethral catheter 6 or 8 times in every other week, the second one is the short effectiveness of the treatment. The most important reason for this is that the IC patients cannot hold their urine for long times, the drug does not contact the bladder enough or it does not reach the sufficient level on the bladder tissue. Therefore the GAG layer repair remains temporary and insufficient.

To this end, nanotechnology is started to be used in in vitro intravesical applications. The most important advantage of this new technique is that it makes the penetration to the bladder tissue easier for the drug to be given to the bladder thanks to its drug carrying system function. However, the carriage and penetration of the drug to the bladder are not single handedly sufficient to make the treatment effectiveness last long.

There are a few intravesical agents which are used in routine clinical applications for interstitial cystitis. When these agents are examined in the light of literature, Dimethyl sulfoxide (DMSO) is the only agent among these drugs that has FDA approval. It has been shown that the intravesical application of DMSO provides objective and subjective benefits to the patients and is more effective compared to placebo. It is applied weekly for 6 weeks of cure. There are studies showing that intravesical glycosaminoglycans such as heparin, hyaluronic acid, chondroitin sulfate, pentosan polysulfate are beneficial. The usage of these agents are limited by the lack of prospective randomized studies, the insufficient effectiveness of these drugs and the abundance of their side effects.

There is limited information in the literature on interstitial cystitis model and nanotechnology. When we look at these rare studies, it is seen that different types of nanoparticles have different effects on the bladder, indeed. The studies are at in vitro level. Seven types of nanoparticles that may affect the bladder are defined as; Mucoadhesive (Chitosan, synthetic polymers), Dendrimers, Protein (gel nanoparticle), Lipid (Liposome), magnetic, Gold Nanoshell, in situ gel systems.

Chitosan which is one of the mucoadhesive nanoparticles is used as a drug (mitomycin C) carrier system in bladder cancer due to its bladder urothelium permeability increasing function.

Protein Nanoparticles (gel, etc.) are used in bladder cancer as paclitaxel carriers.

It is shown that in the interstitial cystitis model of liposome (one of the lipid nanoparticles) which is made by using Protamine Sulphate, it protects urothelium from irritant penetration thanks to the lipid biofilm layer it creates on the bladder surface.

Lastly, the Magnetic Nanoparticles are mostly used in diagnostic MR and in a study it is stated that they played a role as doxorubicin carriers in the bladder.

During the research made in the literature, some applications on the subject were encountered. One of these is the patent application publication No. TR201103387 titled “Pharmaceutical composition comprising quinuclidin-3′-yl 1-phenyl-1,2,3,4,-tetrahydroisoquinoline-2-carboxylate for treatment of interstitial cystitis and/or abacterial prostatitis”. The summary of the invention is as follows: ‘A depressant of capsaicin-sensitive sensory nerve, containing quinuclidine-3′-yl 1-phenyl-1,2,3,4-tetra-hydroisoquinoline-2-carboxylate or a salt thereof as an active ingredient, specifically a therapeutic drug of interstitial cystitis, hypersensitive disorder of the lower urinary tract, and/or abacterial prostatitis.’

Another similar one is the patent application publication No. TR 201203857 titled ‘Pde 4 inhibitors for the treatment of interstitial cystitis’. The summary of the invention is as follows: This invention relates to low molecular weight sodium hyaluronate usage between 120 kdalton and 200 kdalton range, in 50 to 180 mg/50 ml solution, by creating a temporary layer on the bladder wall in the treatment of interstitial cystitis, recurrent bacterial cystitis and radiation cystitis.

As a result, an improvement in the technical field on the treatment method of IC disease has become a necessity due to the insufficiencies of the current solutions on the subject because of the problems mentioned above.

### OBJECTIVE OF THE INVENTION

Present invention relates to the development of magnetic-micro particles that meet the above mentioned necessities, removes all disadvantages and bring some additional advantages, provide controlled Glycosaminoglycan (GAG) release; and the usage of intravesical for interstitial cystitis.

The main objective of the invention is to provide the drugs which are intravesically applied, to reach the bladder tissue easier thanks to micro particles obtained through nanotechnology. Also, by means of the interaction between the Magnetic micro particle which makes GAG release and magnetic tape that is placed in suprapubic area, the drug is intended to stay in the bladder for a long time.

Another objective of the invention is to prevent the drug to be thrown out of the bladder in a short time in interstitial cystitis patients and, to enable the repairing of long term GAG defect as a result of bladder being exposed to more drugs thanks to this carrier system that uses Bioadhesion.

A magnetic tape placed in the suprapubic area and a release mechanism comprising of the drug, which provides a grip in the bladder, has magnetic feature and presents slow release, are used in the invention. This mechanism can be used in all lumen organs and in case any drug is desired to be released for a long time; in all lumen organs, all urinary system in urology, intrauterine applications in gynecology, lumen organs like stomach-intestines in gastroenterology.

It is a method for the repair of damaged glycosaminoglycan layer in the bladder due to interstitial cystitis in order to meet the above mentioned objectives, wherein it comprises of the process step of covering said glycosaminoglycan with magnetic nanoparticles.

Structural and characteristic features and all advantages of the invention will be understood more clearly through the figures and detailed descriptions made with reference to these figures below and therefore the evaluation needs to be carried out by considering these figures and detailed descriptions.

### FIGURES THAT HELP THE INVENTION TO BE UNDERSTOOD BETTER

FIG. 1 is the view of Glycosaminoglycan (GAG) layer defect in interstitial cystitis.

FIG. 2 is the view showing the drug, in the injector, which is prepared with nanotechnology, has magnetic feature and can make slow release.

FIG. 3 is the view of subcutaneous magnetic tape that is placed in the suprapubic area to hold the drug with magnetic feature in the bladder.

FIG. 4 is the view showing the drug spreading on the bladder inner surface in the style of a gel.

FIG. 5 is the view of a healthy bladder.

FIG. 6 is the view of a urethral catheter.

The drawings should not necessarily be scaled and the details that are not necessary to understand the invention might have been neglected. Other than this, the elements that are at least identical to a great extent or that are identical to a great extent at least in terms of their functions are shown with the same numbers.

### EXPLANATION OF PART REFERENCES

1. Kit

10. Bladder

11. Suprapubic area

20. Magnetic tape

21. Suture

30. Urethral catheter

40. Syringe

### DETAILED EXPLANATION OF THE INVENTION

In this detailed explanation, the preferred embodiments of the structuring of development of magnetic-micro particles that provide controlled Glycosaminoglycan (GAG) release and the intravesical usage of it in Interstitial cystitis method according to the invention are described only for the subject to be understood better without any limiting effect.

First of all, the GAG which is covered with magnetic nanoparticles is prepared to be used in the invention.

Preparation Method of Molecule with Nanotechnology

Intravesical Drug: The content of the GAG which is the main substance will be covered with micro particles and prepared in nanotechnology laboratory (it will transform into gel form in the bladder).

Intravesical drug preparation method: Magnetic micro particles that include 5 mg/kg GAG will be introduced into the bladder with a layer by layer method.

Synthesis of Magnetic Micro Particles

In the method of the present invention, the magnetic micro particles will be synthesized with reflux method. A certain amount of Fe³⁺ salt (for example FeCl₃.4H₂O) will be dissolved in 50 ml water in a 3 necked flask, then reflux is applied for 2 hours at 100° C. on heating mantle. The iron solution which is brown in color before the reflux procedure gets a black color at the end of 2 hours and solid particle formation at the bottom of the flask is observed. Then the solid product in the 3 necked flask is separated from the solution using magnetic separation method and is dried for 2 hours at 60° C. after being washed twice with water and once with ethyl alcohol. In order for the surface of the synthesized Fe₃O₄ particle not to be oxidized due to the oxygen in the air and form a Fe₂O₃ layer on the surface, N₂ gas should be passed through the reaction environment throughout the synthesis. The main advantage of this method is that it is a green synthesis method which means that the chemical material used for the synthesis is only Fe³⁺ salt and there is no need for any base solution in order for the precipitation to happen. The sizes of the magnetic particles to be synthesized are aimed to be 1-5 μm. The synthesized magnetic particles will be characterized using FTIR, XRD and TEM methods of analysis.

Covering the Surfaces of the Magnetic Micro Particles with GAG

First, the surfaces of the synthesized magnetic particles will be modified with polyacrylic acid. For this procedure; magnetic particles will be added to the polyacrylic acid solution which will be solved in water and after approximately 6 hours of mixing at 50° C. it will be separated using a magnet and washed. Acquired acid functional particles will be added to first collagen solution (1% water solution) and then GAG solution (1% water solution) using layer by layer method and will be mixed for 3 hours in each solution. After that, after will be washed with water each time, using separation by magnet. Magnetic parts will be covered approximately with 100 layers and then will be dried in room temperature. The release feature of the covered polymer layers will be gravimetrically applied to the acquired particles developing solutions similar to time based urine solution. After determining the most appropriate number of layers in vivo studies will be made.

Said invention consists of two fixed parts:

1. GAG with magnetic micro particles (Drug)

2. A KIT consisted of a combination of Magnetic Tapes (20)

Development of magnetic microparticles that can release drug (GAG) in a controlled fashion and holding this molecule in the bladder (10) are provided by the invention. Holding the GAG covered with magnetic particles in the bladder (10) is provided by the magnetic interaction between magnetic tapes (20) that are placed in the suprapubic area (11) and the magnetic particles.

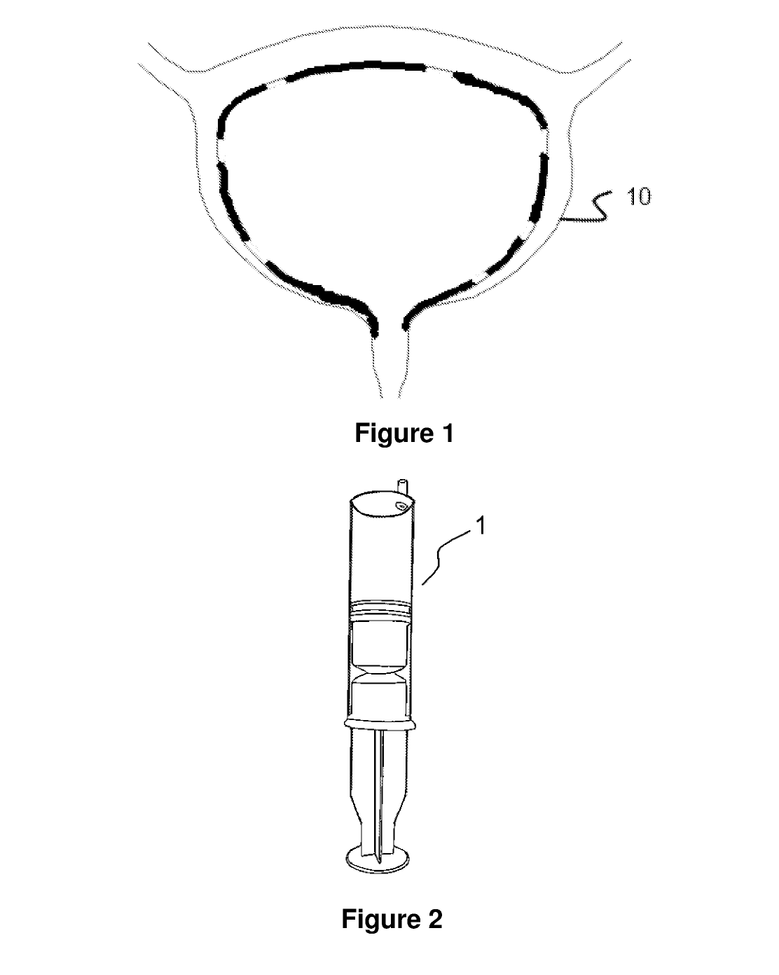

A view of Glycosaminoglycan (GAG) layer defect in interstitial cystitis is illustrated in FIG. 1. When interstitial cystitis is formed, defect is seen in (GAG) layer of the bladder (10). In that case, the kit (GAG with magnetic micro particles) application that might be positioned in the place of the layer that disappeared is made as follows;

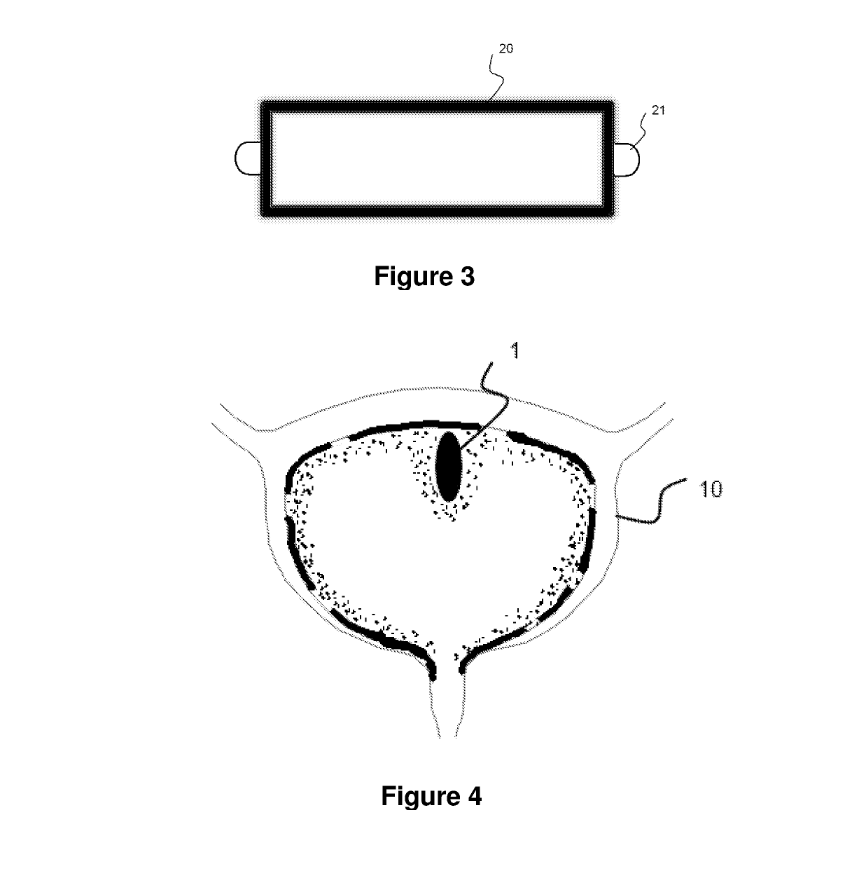

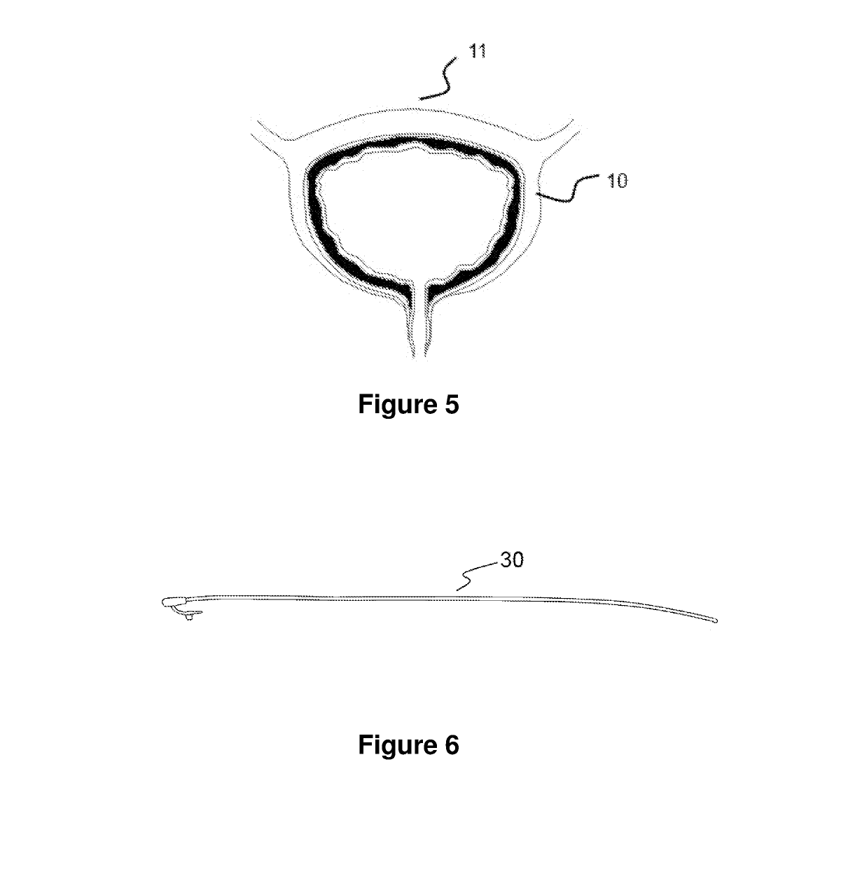

In FIG. 3 there is the view of subcutaneous magnetic tape (20) that is placed in suprapubic area (11) for the GAG which is covered by magnetic featured nanoparticles to get a grip in the bladder (10). In FIG. 4 there is the view of the kit (1) spreading in gel style to the inner surface of the bladder (10). First, GAG with magnetic micro particles is prepared as mentioned above in detail. Then magnetic tape (20) is placed in suprapubic area (11). The reason for the placement of magnetic band (20) in suprapubic area (11) is to ensure the GAG with magnetic micro particles get a grip in the bladder (10) and spread homogeneously. After the magnetic tape (20) is placed in the suprapubic area (11), it is fixed via suture (21). Then, via syringe, the prepared GAG with magnetic micro particles is introduced into the bladder (10) with the help of 1OF urethral catheter (30). When the GAG with magnetic micro particles is injected in the bladder (10); it interacts with the magnetic tape (20) that is placed in the suprapubic area (11) and spreads in the bladder (10) providing it to hold for a long time in the bladder (10). Therefore the interstitial cystitis patients are aimed to need the treatment in less frequent periods.

Thanks to this kit (1) which can stay in the bladder (10) for a long time and release slowly, the treatment method that does not require frequent intravesical application is defined in interstitial cystitis (IC) patients.

1. A method for the repair of damaged Glycosaminoglycan layer in the bladder due to interstitial cystitis wherein said Glycosaminoglycan comprises of the process step of covering with magnetic nanoparticles.

2. The method in accordance with claim 1, wherein said magnetic micro particles are synthesized with the reflux method.

3. The method in accordance with claim 2, wherein said reflux method comprises process step of reflux application for 2 hours at 100° C. on heating mantle after getting Fe³⁺ salt and dissolving it in a flask with the help of water.

4. The method in accordance with claim 3, wherein the iron solution which is brown in color before the reflux procedure gets a black color at the end of 2 hours and solid particle formation is observed at the bottom of the flask.

5. The method in accordance with claim 4, wherein the solid product in the flask from is separated by using magnetic separation method.

6. The method in accordance with claim 5, wherein the product separated from the solution by using magnetic separation method is dried for 2 hours at 60° C. after being washed with water and ethyl alcohol.

7. The method in accordance with claim 6, characterized in that N₂ gas is passed through the reaction environment throughout the synthesis in order for the surface of the synthesized Fe₃0₄ particle not to be oxidized due to the oxygen in the air and form a Fe₂0₃ layer on the surface.

8. The method in accordance with claim 7, wherein the sizes of synthesized magnetic parts are 1-5 μm.

9. The method in accordance with claim 8, wherein the surface of the synthesized magnetic particles is modified with polyacrylic acid first.

10. The method in accordance with claim 9, wherein said modification procedure with polyacrylic acid comprises of the process steps of:
adding magnetic particles to polyacrylic acid solution which is dissolved in water and mixing them,
after mixing, separating them via magnet and washing.

11. The method in accordance with claim 10, wherein by using layer by layer method the acquired acid functional particles comprise of the process steps of:
adding them first to collagen solution,
then to GAG solution and mixing them for 3 hours in each solution.

12. The method in accordance with claim 11, wherein they are washed with water each time, by using separation by magnet method after said mixing procedure.

13. A kit to be used for the repair of Glycosaminoglycan layer which is damaged in the bladder due to interstitial cystitis, characterized in comprising:
Glycosaminoglycan covered with magnetic nanoparticles mentioned in claim 1,
subcutaneous magnetic tape that interacts with magnetic nanoparticle which releases Glycosaminoglycan, and that is placed in the suprapubic area in order for said Glycosaminoglycan to get a grip in the bladder and make it stay in the bladder for a long time.

14. The kit mentioned in claim 13, wherein said magnetic tape is fixed to suprapubic area via suture.

15. The kit mentioned in claim 13, wherein said Glycosaminoglycan covered with magnetic nanoparticles is introduced into the bladder via a syringe.
